(19) United States
(12) Patent Application Publication
(10) Pub. No.: US 2026/0245200 A1
(43) Pub. Date: Aug. 20, 2026

(54) METHODS OF RADIOMICS FEATURES EXTRACTION

(71) Applicant: SOPHiA GENETICS S.A., Rolle (CH)

(72) Inventors: Thierry COLIN, Bordeaux (FR); Loic FERRER, Bordeaux (FR); Olivier GALLINATO, Bordeaux (FR); Jason SIFFRE, Rolle (CH); Floriane GIDEL, Rolle (CH)

(73) Assignee: SOPHiA GENETICS S.A., Rolle (CH)

(21) Appl. No.: 18/871,392

(22) PCT Filed: Jun. 2, 2023
(86) PCT No.: PCT/EP2023/064896

§ 371 Date: Dec. 3, 2024

(30) Foreign Application Priority Data
Jun. 3, 2022 (EP) 22290038.3

(51) Int. Cl. G06T 7/00; G06V 10/26; G06V 10/44; G06V 10/70

(52) U.S. Cl. CPC G06T 7/0012; G06V 10/26; G06V 10/457; G06V 10/70; G06T 2207/10116; G06T 2207/20036; G06T 2207/30056; G06T 2207/30061; G06T 2207/30096; G06V 2201/03

(57) ABSTRACT

A multi-purpose automated radiomics workflow is proposed which facilitates the extraction of a bank of radiomics features for a diversity of imaging modalities and patient pathologies. It is versatile enough to provide a robust extraction of the radiomics features without requiring manual corrections even when the patient pathology is characterized by multisite lesions and thus multiple segmented sites, such as for instance multiple tumors or metastasis sites in late cancer stages.

### FIELD

The present invention relates to the field of medical imaging data processing. In particular, the present invention relates to the deep learning predictive models of a radiomics signature for patients and to the computer-implemented methods of extracting and refining radiomics features from medical images.

### BACKGROUND

Emerging patient care data processing platforms enable the generation of high-throughput quantitative imaging features for a diversity of medical applications in clinical practice. This features can be used to support a number of clinical applications from diagnosis to the choice of treatment and prognosis, in particular the increasing medical demand for personalized medicine in oncology. In this context, radiomics has emerged as a discipline for helping clinicians move beyond limited medical imaging gold standard response criteria. Streamlined, end-to-end, automated radiomics workflows aim at segmenting medical images, extracting standardized radiomics features and correlating them with outcomes.

In this context radiomics research is booming—in 2020, more than 1,500 peer-reviewed radiomics papers were published (Pinto dos Santos et al. (2021), European Radiology, 31, pp 1-4). In oncology, the use of a radiomic signature based on the extraction of radiomics features from patient medical imaging have recently been shown to facilitate the prediction of efficacy of immunotherapy in lung cancer (Sun et al (2018), The Lancet Oncology, Vol. 19 (9): 1180-1191; Tunali et al. (2019), J Thoracic Oncology Vol. 14 (11) Sup 1 S1129; Mu et al. (2020), Eur J Nucl Med Mol Imaging 47 (5): 1168-1182). Given the importance of radiomics for a diversity of clinical applications, initiatives have recently emerged to standardize the radiomic analysis and features extraction, such as the Imaging Biomarker Standardization Initiative (IBSI—https://theibsi.github.io/and Zwanenburg et al. (2016)—eprint arXiv: 1612.07003). These standards rely upon the computer-implemented extraction of several hundreds of radiomics features such as features related to:
morphological features characterizing the shapes of objects of interest, for instance area, volume, compactness, or sphericity of tumor objects in the images;
first order features such as luminance and color intensity histograms;
second-order statistics features such as textural features, for instance GLCM (Grey Level Co-occurrence Matrix) or Haralick features;
as well as higher order statistical features extracted by various signal processing methods from the image data, for instance digital signal filters and frequency domain transforms to better characterize certain image patterns.

The feature extraction image analysis and image processing algorithms can be implemented in medical imaging software platforms, taking as input the medical imaging data to produce a bank of radiomic features extracted from the medical images. A radiomics workflow to extract a bank of features from segmentation in imaging data in combination with machine learning classifiers such as decision trees and support vector machines (SVM) was described in WO2007/079207. Fornacon-Wood et al. European Radiology (2020) 30:6241-6250, reported 14 radiomics software platforms cited in literature, including the widely used pyradiomics open-source python package (https://pyradiomics.readthedocs.io/en/latest/index.html and van Griethuysen et al. (2017) Cancer Research, 77 (21), e104 e107) and other open-source packages. However, there is no golden standard way of ensuring reliability and harmonization of the features calculations due to the multiple modes of parametrizing these algorithms. More recently, the use for more specific applications such as oncology of advanced automated AI methods such as those based on deep learning predictive models (DL) was investigated. It was shown that integration of this methods as part of end-to-end radiomics workflow allows to robustly select most relevant radiomics features. Papadimitroulas et al., Physica Medica 83 (2021), 108-121 provides a review of radiomics extraction and neuron networks DL models for image analysis in oncological radiomics. These models enable two major improvements over the prior art:
1) to refine the extraction of multiple radiomics features from input medical images, in particular by using DL models for image segmentation (instead of a manual or semi-automated segmentation);
2) to select the most relevant features (robust radiomics features) suitable for a given purpose, such as the prediction of a specific treatment response.

In clinical practice, the medical team may wish to evaluate different treatment options, for instance immunotherapy treatments and other treatments, based on the patient omics, including but not limited to the patient radiomics features. Prediction models are developed specifically for a given application (e.g. a given treatment), and recent work use different subsets of the radiomics features extracted from the patient imaging data: for instance, the radiomics features developed Sun et al (2018) are not the same as the radiomics features developed by Mu et al. (2020). Not only the radiomics features may change in the absence of established standards, but even the input imaging data used for extracting the radiomics features may vary according to the application for instance US2020/0000396 proposes to jointly analyze MRI imaging with H&E imaging from a prostate cancer patient to classify patients between low and high DECIPHER risk groups after radical prostatectomy. Another example of a multi-omics application combining CT image radiomics features and laboratory indices is described in Tang et al., Severity assessment of COVID-19 using CT image features and laboratory indices, Phys. Med. Biol. 66 (2021) 035015.

In order to optimize the cost of the imaging data processing and storage infrastructure for medical centers along the patient care journey, there is also a need for radiomics extraction systems and workflows which can produce and store a bank of radiomics features to be later retrieved and used by an application specific solution. In some applications, for instance in multi-omics medical data analysis and/or longitudinal follow-ups of a patient over multiple evaluation time points along his/her care journey, this avoids to repeat the whole radiomics features extraction process from the patient imaging data in the PACS system when the medical team is looking into new predictions from the integration of the former radiomics data in the multi-omics and/or longitudinal analysis.

There is also a need to prepare radiomics features for integration into a fully automated radiomics system and workflow as part of a personalized medicine omics platform which facilitates the diagnosis, the prognosis, the prediction of the treatment response and/or the prediction of the treatment efficacy over time for a specific patient, based on his or her medical imaging data characteristics, while optimizing the resource allocation spend of healthcare systems.

Another emerging problem is the increasing demand for longitudinal evaluations of the patient pathology evolution at different times along the patient healthcare journey. There is therefore a further need for more radiomics features extraction and storage which allow later retrieval and differential comparisons of longitudinal imaging for a given patient, even in the most challenging advanced cancer stages with new multisite lesions to characterize in the later stage imaging data.

A further emerging problem raised by the development of automated radiomics workflows is the difficulty to extract, store and retrieve universal radiomics features which are able to accurately represent radiological images regardless of whether they comprise only simple or more complex pathological regions spread over multiple lesions which may appear as disjoint in the radiological images, while sharing the same pathological origin. For instance, morphological features from the IBSI or pyradiomics sets have been developed for the simplest cases and do not capture properly the topological complexity of many pathologies in particular in advanced stages. The feature extraction therefore requires manual feature sorting (e.g. filtering out non-relevant morphological features, at the risk of losing key information that is visible with the expert eye) or manual segmentation data processing (e.g. forcing the segmentation data to consider only the most relevant site, at the risk of losing key information from secondary pathological regions). These manual pre- or post-processing steps cause lack of accuracy in the bank of radiomics features data. Moreover, they do not scale well with the increasing automation and deployment of data-driven medicine systems and platforms. There is therefore a need for a more accurate automated radiomics feature extraction and storage method which can be fully computer-implemented and which can apply indifferently to simple or complex radiological images without requiring manual intervention or correction.

### SUMMARY

The present invention is based on the development of a particular computer-implemented method for producing a bank of radiomics features from the radiological imaging data of a patient pathology having multiple lesion sites, wherein data from all lesion sites are taken into consideration and processed accordingly. In the methods, processed are radiological imaging data that are corresponding to non-neighboring areas which are geometrically disjunct in the image data yet topologically and/or functionally connected as belonging to the same object of interest (i.e., a pathology localized in an organ, e.g., tumor(s) in a lung). The image components corresponding to these non-neighboring areas of the same object of interest are known as connected components. The methods of the invention comprise a step of extracting a bank of radiomics features from the connected components with a step of producing at least two values selected from: a total value, an average value and a maximum value of at least one radiomics feature calculated from all the connected components. This step allows to extract a bank of radiomics features from the radiological imaging data of all the sites from a pathology having multiple lesion sites. The extracted bank of radiomics features may be for further use in any known method of analyzing patient state, disease progress and the like. Thus, obtaining a bank of radiomics features from the radiological imaging data of a pathology having multiple lesion sites allows for better/more detailed/more accurate further analysis.

Further, the methods of the invention are particularly suitable for processing the radiological imaging data of a patient pathology having multiple lesion sites, wherein the methods are automated. Automation allows for further standardization and acceleration of the process and removes possible operator bias. The methods of the invention are particularly suitable for processing the radiological imaging data of a patient pathology having multiple lesion sites, wherein the methods are automated and comprise the use of a segmentation model trained to extract, from radiological imaging data, at least two connected components.

In one embodiment provided is a computer-implemented method for producing a bank of radiomics features from the radiological imaging data of a patient pathology having multiple lesion sites, the method comprising the steps of:
a) receiving radiological imaging data of the patient from a database of medical data;
b) segmenting the radiological imaging data to produce at least two connected components, each connected component characterizing a lesion site, the connected components forming an object of interest to characterize a pathology of the patient;
c) extracting a bank of radiomics features from the at least two connected components;
d) storing the bank of radiomics features in a database of digital health features;
   characterized in that:
extracting a bank of radiomics features comprises a step of producing at least two values selected from: a total value, an average value and/or a maximum value of at least one radiomics feature calculated from all the connected components.

In further embodiment provided is the computer-implemented method according to the invention, further comprising a step of localizing, from the radiological imaging data, at least one anatomical area of interest to produce an anatomical area label. In further embodiment, this step of localizing is comprising the steps of acquiring a localization model trained to extract an anatomical area label from radiological imaging data and applying the localization model to the radiological imaging data to produce an anatomical area label.

In further embodiment provided is the computer-implemented method according to the invention, further comprising a step of identifying, from the radiological imaging data, a set of imaging signal properties. In further embodiment, this step of identifying is comprising the steps of acquiring a signal identification model trained to extract a set of imaging signal properties from radiological imaging data and applying the signal identification model to the radiological imaging data to produce a set of imaging signal properties.

In further embodiment provided is the computer-implemented method according to the invention, further comprising the step of selecting, for each anatomical area label, a segmentation method for segmenting the radiological imaging data according to the imaging signal properties. In one embodiment the segmentation method is a manual method, a semi-automated method, or an automated method. In further embodiment, the segmentation method is an automated method, comprising the steps of acquiring a segmentation model trained to extract, from radiological imaging data, at least two connected components and applying the segmentation model to the radiological imaging data to produce at least two connected components. In one embodiment, the segmentation method produces a set of at least two connected components and wherein extracting a bank of radiomics features from the connected components comprises aggregating the at least two connected components; calculating a radiomics feature value from the aggregated connected components to produce a summarized radiomics feature value; storing the summarized radiomics feature value into the bank of radiomics features. In another embodiment, the segmentation method produces a set of at least two connected components and wherein extracting a bank of radiomics features from the connected components comprises calculating a component-specific radiomics feature value from each component in the at least two connected components; averaging the component-specific radiomics feature values to produce an average feature value; storing the average radiomics feature value into the bank of radiomics features.

In another embodiment, the segmentation method produces a set of at least two connected components and wherein extracting a bank of radiomics features from the connected components comprises measuring a size of each connected component in the at least two connected components to identify the largest connected component; calculating a component-specific radiomics feature value from the largest connected component; storing the component-specific radiomics feature value into the bank of radiomics features.

In further embodiment provided is the computer-implemented method according to the invention, wherein at least one of the radiomics features is a morphological feature, wherein the segmentation method produces at least two connected components and wherein extracting a bank of radiomics features from the connected components comprises: aggregating all the connected components in the at least two connected components and calculating a summarized morphological feature from the aggregated connected components to produce a first morphological radiomics feature value; calculating a component-specific morphological feature value from each component in the at least two connected components, and averaging the component-specific radiomics feature values to produce a second morphological radiomics feature value; measuring a size of each connected component in the at least two connected components to identify the largest connected component, and calculating the component-specific morphological feature from the largest connected component to produce a third morphological radiomics feature value; storing the first, second and third morphological radiomics feature values into the bank of radiomics features.

In further embodiment provided is the computer-implemented method according to the invention, wherein the patient pathology is a cancer, and each connected component corresponds to a different tumor or metastasis site. In one embodiment the pathology is a non-small cell lung cancer (NSCLC) at stage IV, the anatomical area of interest is lung, and at least one of the connected components corresponds to a lung tumor or metastasis. In another embodiment the pathology is a non-small cell lung cancer (NSCLC) at stage IV, the anatomical area of interest is liver, and at least one of the connected components corresponds to a liver metastasis.

In further embodiment provided is the computer-implemented method according to the invention, wherein the patient pathology is an infectious disease, the anatomical area of interest is lung and at least one of the connected components corresponds to a different ground glass opacity site. In one embodiment the patient pathology is SARS-Cov2.

In one embodiment provided is the computer-implemented method according to the invention, wherein the radiomics feature is a morphological radiomics feature selected among an IBSI feature or a pyradiomics feature.

### DESCRIPTION OF THE FIGURES

The present description will be better understood with the help of the attached figures, namely:

FIG. 1 shows a radiomics processing system.

FIG. 2 shows a radiomics processing workflow.

FIG. 3a) shows an example of a segmented site from lung cancer imaging data in 2D and FIG. 3b) shows it in 3D.

FIG. 4 illustrates two possible radiomics feature extraction workflows to process multiple segmented sites corresponding to different lesion locations of tumors and metastases as may be observed in some late stage cancers, respectively for a) a non-morphological feature and b) a morphological feature.

FIG. 5a) shows an example of a segmented multi-lesion site from lung cancer imaging data in 2D and FIG. 5b) shows it in 3D.

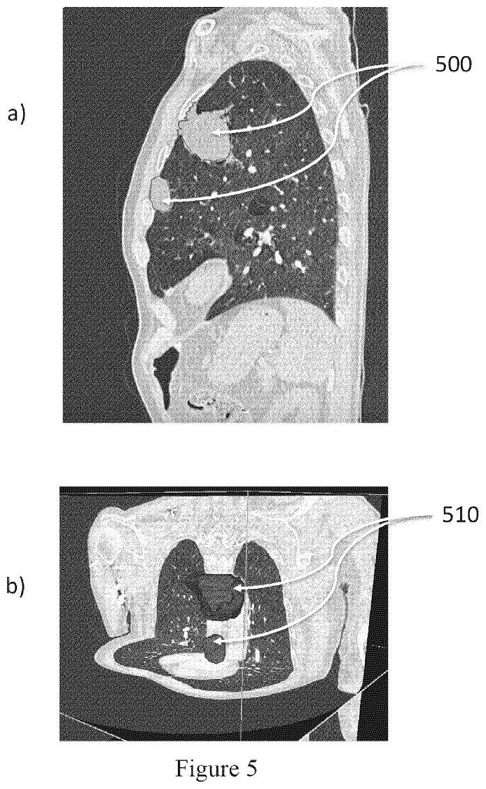

FIG. 6 shows a further example of a radiomics image with two connected components.

### DETAILED DESCRIPTION

The term medical image data or radiological data or imaging data or radiolological imaging data refers to the digital images data, comprising one or more image files and their metadata, as can be collected and archived for a patient at any time point in its health care path journey. These images may be acquired from the patient examination in one or more medical centers in charge with one or more imaging modality such as CT, PET, MRI, SPECT, ultrasound, X-rays, and others. The images may be in 2D or 3D format. These images may be securely collected, stored, archived and transmitted to the radiomics processing system of the invention in accordance with the PACS (Picture Archiving and Communication System) and DICOM (Digital Imaging and Communications in Medicine) digital medical imaging technology standards that are widely deployed in healthcare organizations worldwide. The term radiomics as an abbreviation of radiology omics refers to the high-throughput digital extraction of mineable, quantitative data from radiological imaging data.

The term feature, indicator, descriptor, imaging feature or radiomics feature refers to an imaging biomarker which can be extracted from imaging data as quantifiable summarization of the image, for instance a statistical value. The radiomics features or radiomics descriptors refer to a set of values computed from the segmentation of an image area (2D Region of Interest ROI or 3D Volume of Interest VOI), using the intensity values of the 2D pixels or 3D voxels included in the segmented ROI or VOI site. In a radiomics workflow, multiple features, each representative of a different characteristic of the ROI or VOI segmented site in the image, may be individually extracted with a computer-implemented method and combined to produce a bank of features or a radiomics signature. This bank of features may include, but is not limited to heterogeneity, morphological (0-order), intensity (1st order), texture (2nd order) or higher order features. Example of banks of features commonly used in radiomics include any of the 169 well established features of the IBSIl standard, any of the 1500 features of the Pyradiomics open source software package, and/or any of the LIFEx (www.lifexsoft.org), CERR, or IBEX public software tools. Preferably, radiomics features are represented as scalar values shifted and rescaled so that they fit into the range between 0 and 1 (normalized features), but some radiomics features may also be labels according to a predefined dictionary, or measurements in predetermined units. Various computer-implemented methods to extract various normalized radiomics features from image segmentation data can be used to produce a bank of radiomics features, such as various commercial products or free open source software tools of the public domain as listed for instance in Appendix E3 of Shur et al., Radiomics in oncology: a practical guide, Radiographics Vol. 41 No. 6, Oct. 1 2021. A broadly employed example of such tools is the open-source python package of pyradiomics (https://pyradiomics.readthedocs.io).

The term localization refers to the identification of a target anatomy element, for instance an anatomical area, an organ or part of an organ to be segmented in the medical images. Preferably, localization refers to the identification of an organ or part of an organ, with a digital label suitable for the annotation of the medical images metadata in computer-implemented processing.

The term segmentation refers to the delineation of an object of interest or a site of interest—Region of Interest (ROI) in 2D, Volume of Interest (VOI) in 3D—to extract object shapes from a localized anatomy area. Segmentation may be achieved either manually by an operator; or semi-automatically by an image processing computer-implemented software under closed supervision, configuration and parametrization by an operator (for instance, using the ImageJ software suite tools such as the region based methods, the graph-based methods, the shape-based methods, the morphological methods, the clustering methods, the thresholding methods and others from https://imagej.net); or automatically using a machine learning segmentation model, such as for instance a deep-learning model of image segmentation. In the past decades, many automated segmentation methods used signal processing algorithms applied to digital image representations (Sharma et al., Automated medical image segmentation techniques, Review Article, J. of Med. Physics, Vol. 35, No. 1, 3-14, 2010); more recently, artificial intelligence methods such as deep learning (DL) and in particular convolutional neural networks (CNN) have been more and more applied to provide more robust segmentation models (Papadimitroulas et al., Physica Medica 83 (2021), 108-121, Table 1: Segmentation). In the case of a tumor, segmentation enables to extract all of the various areas inside and around the tumor (also called habitats), which vary with the tumor characteristics such as blood flow, cell density, etc.

The term connected components refers to image components extracted from the image segmentation process as sets of pixels or voxels, corresponding to non-neighboring areas which are geometrically disjunct in the image data yet topologically and/or functionally connected as belonging to the same object of interest. For instance, a tumor region of interest may be formed of multiple separate lesions sharing the same pathological origin. In general, each segment in the segmentation data representation may correspond to a different lesion site. In advanced image processing practice, in line with the mathematical vocabulary from topology theory, different segments collectively forming a multisite tumor region are called connected components. In the case of a metastatic tumor, or more generally in the case of a multisite lesion to characterize in relation with some pathologies, segmentation enables to extract each lesion site to produce one or more connected components collectively forming the object of interest.

The term feature extraction refers to the signal processing analysis and/or calculation of a quantifiable value, such as a measurement or a normalized feature between 0 and 1, from a digital signal input, such as from an image, from an image segment (single connected component), or from at least two connected components collectively representing a segmented object of interest in an image.

### Radiological Imaging Data

Radiological imaging data are images collected and include, but are not limited to CT, PET, PET/CT MRI, SPECT.

Radiological imaging data for the patient may include, but are not limited to the patient's imaging at pre-baseline, at baseline, at first evaluation, or at any further evaluation time in a longitudinal follow-up.

Radiological imaging data for a cancer patient may include, but are not limited to the patient's: millimetric injected CT cancer site scan at portal time, PET/CT, CT, MRI, SPECT.

In one embodiment, the radiological imaging data for a patient with a lung cancer, in particular with stage IV NSCLC, may comprise at least one or consist of the following: millimetric injected CT thoracic, abdomen and pelvis scans at portal time, PET/CT, brain CT, brain MRI; chest CT scan; CT-TAP scan; brain CT scan or PET/CT.

### Radiomics Feature Extraction System

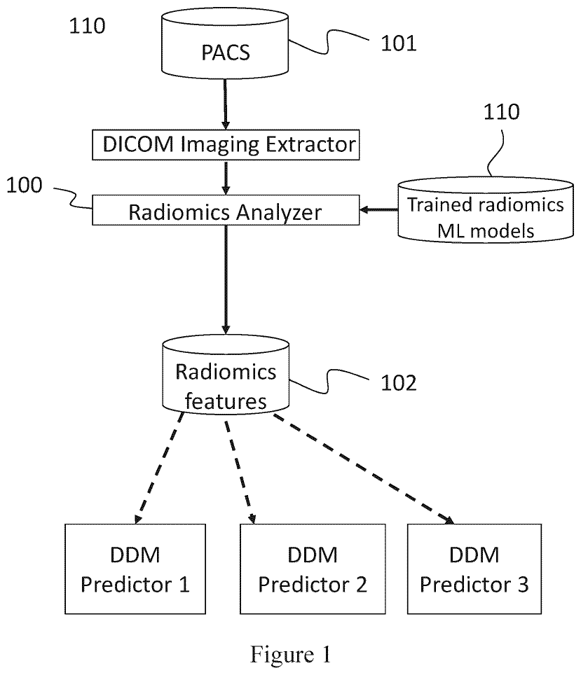

FIG. 1 illustrates a radiomics feature extraction system as may be integrated within an end-to-end patient care omics or multi-omics platform. In a medical center image archival system, possibly as part of an electronic healthcare records (EHR) system, radiological imaging data may be stored in a first database 101 of medical data, for instance in a database of medical imaging data in accordance with the PACS/DICOM standards.

For a given patient identifier (possibly an anonymized identifier), archived radiological imaging data may be extracted from the medical imaging data with a DICOM compliant imaging extractor. The radiological imaging data comprises one or more radiological images and possibly some associated metadata information. The metadata usually comprises at least information on the date of the imaging examination and a patient identifier, preferably anonymized. It may also comprise any first level image property parameters from the imaging examination, such as the imaging modality (MRI, CT), some first level imaging parameters specific to the modality, possibly the imaging equipment manufacturer and version information, and other metadata information directly available from the imaging examination.

The imaging extractor transmits the retrieved radiological imaging data to a radiomics analyzer 100 which extracts radiomics features and stores them as a bank of features for the patient into a second database of digital health features. The bank of radiomics features of a patient may be used by different applications at different times, depending on the patient pathology and status. In particular, the bank of radiomics features may be accessed and used at a later time by a data-driven medicine DDM platform (not represented) to predict, with one or more prediction models, a diagnosis or a prognosis for the patient. DDM Predictors for the patient may include, but are not limited to: a predictor of a diagnosis of a pathology; a predictor of a stage of a pathology; a predictor of a prognosis; a predictor of a treatment response; a predictor of a treatment efficacy. Examples of pathologies may include, but are not limited to: a cancer; a neurological disease; a cardiac disease; a musculoskeletal disease; an infectious disease. The DDM Predictors may employ the radiomics features extracted from the patient's imaging at pre-baseline, at baseline, at first evaluation, or at any further evaluation time in a longitudinal follow-up.

In a preferred embodiment, the Radiomics Analyzer 100 is at least partly automated by employing one or more trained radiomics models 110 to facilitate:
the pre-processing of the medical imaging data, such as annotation, using partial or fully automated localization and/or signal identification;
and/or the analysis of the medical imaging data, such as segmentation of lesion sites of interest.

Examples of AI techniques trained models 110 to facilitate a specific step of the pre-processing and/or the analysis of the medical imaging data are described for instance in Du et al. (2020) Proc. Machine Learning Research 121:174-192 for extraction of localization information from unlabeled DICOM datasets, as well as in the review of Papadimitroulas et al., Physica Medica 83 (2021), 108-121. The Radiomics Analyzer 100 may be a computer system or part of a computer system including a central processing unit (CPU, “processor” or “computer processor” herein), memory such as RAM and storage units such as a hard disk, and communication interfaces to communicate with other computer systems through a communication network, for instance the internet or a local network. Examples of radiomics data analyzer computing systems, environments, and/or configurations include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, graphical processing units (GPU), and the like. In some embodiments, the computer system may comprise one or more computer servers, which are operational with numerous other general purpose or special purpose computing systems and may enable distributed computing, such as cloud computing, for instance in a medical data farm. In some embodiments, the radiomics analyzer 100 may be integrated into a massively parallel system. In some embodiments, the radiomics data analyzer 100 may be directly integrated into a next generation sequencing system.

The Radiomics Analyzer 100 computer system may be adapted in the general context of computer system-executable instructions, such as program modules, being executed by a computer system. Generally, program modules may include routines, programs, objects, components, logic, data structures, and so on that perform particular tasks or implement particular abstract data types. As is well known to those skilled in the art of computer programming, program modules may use native operating system and/or file system functions, standalone applications; browser or application plugins, applets, etc.; commercial or open source libraries and/or library tools as may be programmed in Python, Biopython, C/C++, or other programming languages; custom scripts, such as Perl or Bioperl scripts.

Instructions may be executed in distributed cloud computing environments where tasks are performed by remote processing devices that are linked through a communications network. In a distributed cloud-computing environment, program modules may be located in both local and remote computer system storage media including memory storage devices.

It is thus understood that methods described herein are computer-implemented methods.

### Radiomics Features Extraction Workflow

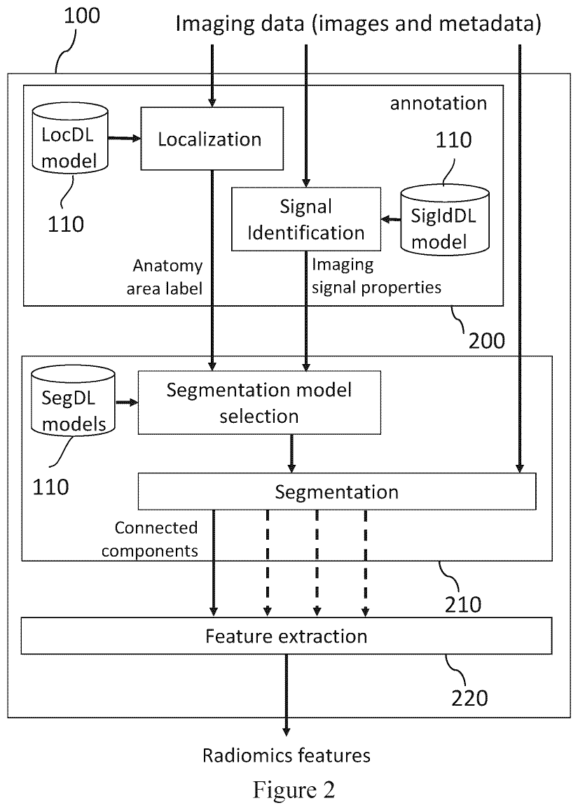

FIG. 2 shows a possible radiomics feature extraction workflow for extracting radiomics features of a patient according to some embodiments of a semi-automated or fully automated radiomics analyzer 100 as described herein.

In one embodiment, is provided a computer program comprising instructions which, when the program is executed by a computer, cause the computer to carry out the method of extracting radiomics features of a patient as described herein.

In one embodiment, is provided a computer-readable medium comprising instructions which, when executed by a computer, cause the computer to carry out the method of extracting radiomics features of a patient as described herein.

In one embodiment, the radiomics analyzer 100 receives, from a first database of medical data, radiological imaging data comprising medical images and metadata from a patient examination. The patient radiological imaging data is first processed by an annotation module 200 adapted to extract an anatomy area label and a set of imaging signal properties from the patient radiological imaging data. The patient radiological data images are then segmented by a segmentation module 210 to extract one or more connected components corresponding to an object of interest in the images. Finally, the radiomics analyzer extracts 220 a set of radiomics features from the connected components.

The annotation module may employ a trained machine learning model 110, preferably a deep learning model (LocDL), to automatically identify the anatomy area label. As will be apparent to those skilled in the art of radiomics machine learning, the machine learning model may be trained using a predetermined set of images for which manually annotated anatomy area labels are available (for instance lung, liver, kidney, brain). The anatomy area label may also further comprise information on the patient orientation and view and/or the scan target and coverage.

The annotation module may employ a trained machine learning model 110, preferably a deep learning model (SigIdDL), to automatically identify the set of imaging properties. As will be apparent to those skilled in the art of radiomics machine learning, the machine learning model may be trained using a predetermined set of images for which manually annotated imaging signal properties are available. In a possible embodiment, the imaging signal properties may comprise:
the 1^{st} level imaging signal parameters such as the imaging modalities and sequences (CT, MRI, PET), as the latter are not always properly documented in a standard metadata format in DICOM systems;
and/or the 2^{nd} level imaging signal parameters such as for instance the use of a CT hard or soft filter, the presence of a contrast agent for MRI or CT imaging, the time of acquisition (arterial, portal, washout), the voxel sizes, the slice spacing, the identification of an MRI T1 or T2 weighting

In a radiomics analyzer workflow, the segmentation module 210 may employ various segmentation methods to segment the patient radiological images. In a possible embodiment, the segmentation module may select a trained machine learning model 110, preferably a deep learning model (SegDL), to automatically segment the patient radiological images. As will be apparent to those skilled in the art of radiomics segmentation, there is no universal segmentation model adapted to multiple anatomy areas and to the diversity of imaging signal properties as may be identified from an automated annotation workflow. Therefore, the segmentation module 210 may rather comprise a library of segmentation models 110, each corresponding to a specific anatomy area in combination with a specific set of imaging signal properties. As will be apparent to those skilled in the art of radiomics machine learning, each machine learning segmentation model may then be trained using a predetermined set of images covering the same anatomy area and captured with the same set of imaging properties. The segmentation module 210 may then select the segmentation model which matches the localized anatomy area label and the set of imaging properties as extracted from the annotation module 200, and segment, with the selected segmentation model, the patient radiological images to produce one or more connected components.

In a possible embodiment (not represented), the segmentation module 210 may also identify that there is no suitable trained machine learning model available in the library of machine learning models 110 matching the anatomy area and the set of imaging properties. The radiomics analyzer may then select a manual or a semi-automated segmentation method and ask for manual or semi-manual supervision from a user, using the radiomics analyzer user interface.

### Features Extraction

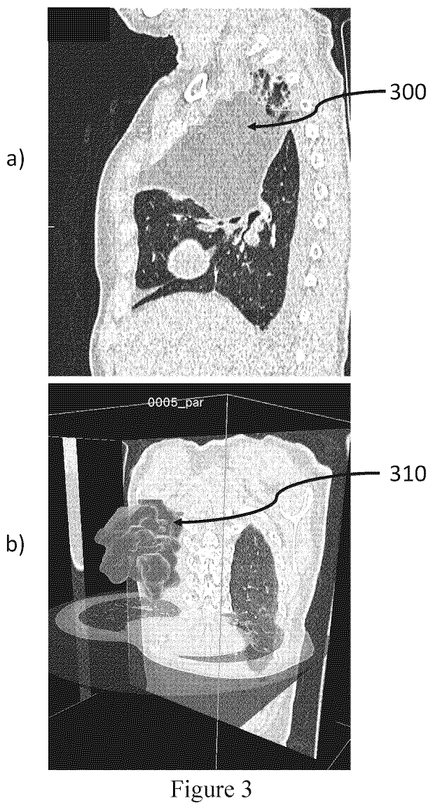

FIG. 3 shows an example of a 2D lung cancer image and the corresponding 3D reconstruction image comprising a single tumor lesion 300, 310. In this example, the segmented object of interest is a tumor and may be represented as a ROI (2D) 300 and reconstructed as a VOI (3D) 310 for display to a user using a graphical user interface. In the automated radiomics analyzer 100 workflow, the object of interest may be represented as a set of at least two connected components, each corresponding to a different lesion site. As will be apparent to those skilled in the art, when a single connected component is extracted from segmentation, prior art methods of feature extraction, for instance as implemented in a conventional IBSI compliant workflow and/or in a free-software tool as listed for instance in Shur et al., may be employed to calculate various radiomics features and produce a bank of radiomics features extracted from the patient radiological imaging data. However, when multiple connected components are present out of the segmentation, the conventional feature extraction workflows process them globally to produce radiomics features for the totality of them. In a possible embodiment, the radiomics analyzer 100 extracts 220 one or more radiomics features from the connected components to produce a bank of radiomics features. The radiomics analyzer 100 may then store the extracted bank of radiomics features in a database 102 of digital health features for the patient.

In one embodiment, the radiological imaging data include data on multiple lesion sites. Each radiomics feature may be computed for the patient pathology by calculating the feature globally as a summarized feature from the multiple connected components. Alternately, each radiomics feature may also be computed from the individual calculation of the radiomics features for each segmented site of the patient pathology lesions separately.

In general, it is useful to use separate morphological descriptors when treating a multi-site annotation as the morphological features have been developed in research for the characterization of the morphology of per-site segmented object shapes (major, minor and least axes for instance). In one embodiment, morphological features are extracted by different methods to better describe the morphological state of a group of lesions (multi-site segmentation), such as selected from, but not limited to:
1. Total feature: The feature is computed for the whole multi-site lesion, by aggregating the connected components into a single component prior to feature calculation.
2. Mean feature: The feature is computed for each connected component individually, then, it is averaged.
3. Max feature: The size of each connected component is measured (for instance, as the connected component area, or its largest diameter, or its volume, or its intensity in a PET scan), and only the feature computed from the largest (volume-wise) site is used.

In one embodiment, all the radiomics features are computed for each of the three above-mentioned categories (total feature, mean feature and max feature).

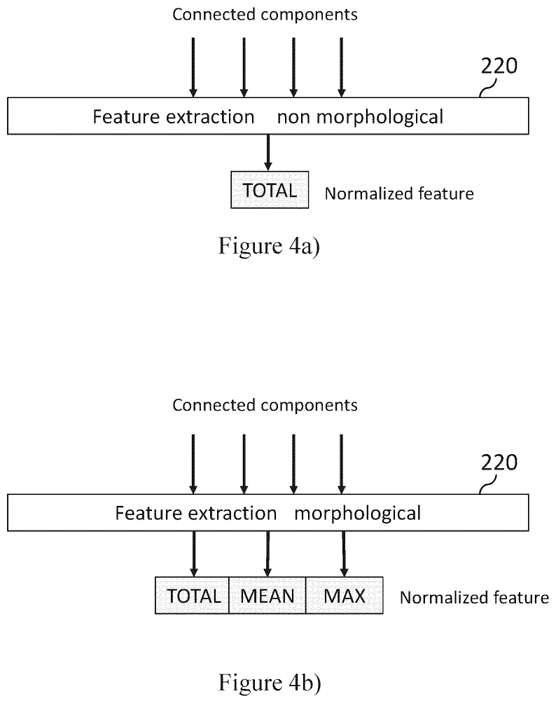

In another embodiment, as illustrated by FIG. 4, for non-morphological features (FIG. 4a) only the total feature is calculated, while for morphological features (FIG. 4b) all the radiomics features are computed for each of the three above-mentioned categories (total feature, mean feature and max feature).

Thus, when there is only a single lesion site in the segmentation data, the above three values are simply computed as the same values; but when there are multiple lesion sites in the radiological images (represented as two or more connected components in the segmentation data), computing these three values results in three different numbers. These three values thus better characterize the pathology than the single, global value of the prior art workflows.

In another embodiment, for non-morphological features only the total feature is calculated; for some morphological features the mean feature and the max feature are computed in addition to the total feature; for some other morphological features where the total feature does not capture meaningful information, the mean feature and the max feature are computed instead of the total feature.

An example of a morphological feature which is better characterized by the computation of its total, mean and max values in the case of multiple lesion sites is the Mass Center Shift feature, defined by the IBSI standard (IBSI code KLMA) as the distance in cm between the ROI volume centroid and the intensity-weighted ROI volume. With the proposed embodiment, the following three values are calculated for this IBSI feature:
total value: the distance between the ROI volume centroid and the intensity-weighted ROI volume, where the ROI is formed of the aggregated multiple lesion sites (note this corresponds to the default KLMA IBSI code value);
mean value: the averaged distance between each lesion site volume centroid and the intensity-weighted lesion site volume (newly introduced calculation);
max value: the distance between the largest lesion site volume centroid and the intensity-weighted largest lesion site volume (newly introduced calculation).

An example of a morphological feature which is better characterized by the computation of its mean and max values while its total value is meaningless in the case of multiple lesion sites is the Surface to volume ratio feature, defined by the IBSI standard (IBSI code 2PR5) as the Ratio between area and the volume of the mesh. With the proposed embodiment, the following two values are calculated for this IBSI feature:
mean value: the average of the ratios between the area and the volume of each lesion site;
max value: the ratio between the area and the volume of the largest site.

Other embodiments are also possible. Table 1 lists a subset of general and morphological features of an extended bank of radiomics features, adapted from the IBSI standard (as identified by an IBSI code in the 5^{th} column), with additional feature values (not indicated with an IBSI code in the 5^{th} column). By adding max and/or average value calculations to the prior art IBSI features, more accurate automated radiomics feature extraction and representation can be obtained in the case of radiological images of pathologies with multiple lesion sites.

The bank of radiomics features produced by the radiomics feature analyzer 100 may then comprise the aggregated global (IBSI default value), as well as the max and mean feature values for one or more of the radiomics features records, in particular for the morphological features.

TABLE 1
Exemplary structure of the general and morphological features of an extended bank of radiomics features, based on the
IBSI standard (as indicated by an IBSI code), with additional feature values (not indicated with an IBSI code) for more
accurate automated radiomics feature extraction and representation of radiological images with multisite lesions.

| | | | | IBSI | |
|---|---|---|---|---|---|
| | | | | CODE (not | |
| IBSI | Abbreviated | Mnemonic | IBSI name (not | IBSI if | |
| Category | group name | name | IBSI if empty) | empty) | Interpretability |
| General | General | Voxel Number | | | Number of voxels within the ROI |
| features | | Positive Voxel | | | Number of voxels with positive |
| | | Number | | | intensity value within the ROI |
| | | GL number | | | Number of discrete intensity values |
| | | | | | within the non-discretized ROI |
| | | GL number after | | | Number of different grey level values |
| | | quantization | | | represented within the discretized ROI |
| | | Number of sites | | | Number of disconnected mesh sites |
| | | | | | within the whole ROI. |
| Morphological | Morpho | Volume (cm³) | Volume | RNU0 | Volume of the mesh |
| features | | Mask volume (cm³) | Mask volume | YEKZ | Volume of the mask |
| HCUG | | Positive Mask | | | Volume of the positive voxels within |
| | | Volume (cm³) | | | the mask. |
| | | Surface Area (cm²) | surface area | C0JK | External surface of the mesh. |
| | | Surface to volume | Surface to volume | 2PR5 | Ratio between area and the volume of |
| | | ratio | ratio | | the mesh |
| | | Compactness 1 | Compactness 1 | SKGS | Measures how compact or sphere-like |
| | | | | | the volume is. |
| | | Compactness 2 | Compactness 2 | BQWJ | Measures how compact or sphere-like |
| | | | | | the volume is. |
| | | Spherical | Spherical | KRCK | Measures how compact or sphere-like |
| | | disproportion | disproportion | | the volume is. |
| | | Sphericity | Sphericity | QCFX | Measures how compact or sphere-like |
| | | | | | the volume is. |
| | | Asphericity | Asphericity | 25C7 | Measures how compact or sphere-like |
| | | | | | the volume is. |
| | | Mass Center shift | Centre of mass | KLMA | The distance between the ROI volume |
| | | (cm) | shift | | centroid and the intensity-weighted |
| | | | | | ROI volume |
| | | Slice maximum 2D | | | The maximum 2D diameter is the |
| | | diameter (cm) | | | maximum distance between the two |
| | | | | | most distant points of the mesh |
| | | | | | projected on each slice of the image |
| | | | | | grid. |
| | | Maximum 3D | Maximum 3D | L0JK | The maximum 3D diameter is the |
| | | diameter (cm) | diameter | | distance between the two most distant |
| | | | | | vertices in the ROI mesh vertex set |
| | | Major axis length | Major axis length | TDIC | The major axis length is twice the |
| | | (cm) | | | largest semi-axis length of the |
| | | | | | ellipsoid, determined using the largest |
| | | | | | eigenvalue obtained by PCA on the |
| | | | | | mask voxels. |
| | | Minor axis length | Minor axis length | P9VJ | The minor axis length is twice the |
| | | (cm) | | | second largest semi-axis length of the |
| | | | | | ellipsoid, determined using the second |
| | | | | | largest eigenvalue obtained by PCA |
| | | | | | on the mask voxels. |
| | | Least axis length | Least axis length | 7J51 | The minor axis length is twice the |
| | | (cm) | | | smallest semi-axis length of the |
| | | | | | ellipsoid, determined using the |
| | | | | | smallest eigenvalue obtained by PCA |
| | | | | | on the mask voxels. |
| | | Inverse Elongation | Elongation | Q3CK | Measures the extent to which a |
| | | | | | volume is longer than it is wide. |
| | | Inverse Flatness | Flatness | N17B | Measures the extent to which a |
| | | | | | volume is flat relative to its length. |
| | | Volume density - | Volume density | PBX1 | Measures the proportion of the mesh |
| | | aligned bounding | (axis-aligned | | axis-aligned bounding box volume |
| | | box | bounding box) | | that is filled by the ROI volume. |
| | | Area density - | Area density (axis- | R59B | Measures the ratio between the ROI |
| | | aligned bounding | aligned bounding | | surface area and the axis-aligned |
| | | box | box) | | bounding box surface area. |
| | | Volume density - | Volume density | ZH1A | Measures the proportion of the mesh |
| | | oriented bounding | (oriented minimum | | oriented bounding box volume that is |
| | | box | bounding box) | | filled by the ROI volume. |
| | | Area density - | Area density | IQYR | Measures the ratio between the ROI |
| | | oriented bounding | (oriented minimum | | surface area and the oriented bounding |
| | | box | bounding box) | | box surface area. |
| | | Volume density - | Volume density | 6BDE | Measures the proportion of the mesh |
| | | enclosing ellipsoid | (approximate | | approximate enclosing ellipsoid |
| | | | enclosing | | volume that is filled by the ROI |
| | | | ellipsoid) | | volume. |
| | | Area density - | Area density | RDD2 | Measures the ratio between the ROI |
| | | enclosing ellipsoid | (approximate | | surface area and the approximate |
| | | | enclosing | | enclosing ellipsoid surface area. |
| | | | ellipsoid) | | |
| | | | Volume density | SWZ1 | Measures the proportion of the mesh |
| | | | (minimum volume | | minimal enclosing ellipsoid volume |
| | | | enclosing | | that is filled by the ROI volume. |
| | | | ellipsoid) | | |
| | | | Area density | BRI8 | Measures the ratio between the ROI |
| | | | (minimum volume | | surface area and the minimal |
| | | | enclosing | | enclosing ellipsoid surface area. |
| | | | ellipsoid) | | |
| | | Volume density - | Volume density | R3ER | Measures the proportion of the mesh |
| | | convex hull | (convex hull) | | convex hull volume that is filled by |
| | | | | | the ROI volume. |
| | | Area density - | Area density | 7T7F | Measures the ratio between the ROI |
| | | convex hull | (convex hull) | | surface area and the convex hull |
| | | | | | surface area. |
| | | Integrated intensity | Integrated | 99N0 | The average intensity in the ROI, |
| | | | intensity | | multiplied by the volume. |
| | | | Moran s I index | N365 | Moran s I index is an indicator of |
| | | | | | spatial autocorrelation |
| | | | Geary s C | NPT7 | Geary's I index is an indicator of |
| | | | measure | | spatial autocorrelation |
| Multiple | MultiMorpho | Volume (cm³) | | | Volume of the mesh |
| lesion sites | | Volume of the | | | Volume of the largest site |
| morphological | | largest site (cm³) | | | |
| features | | Mask volume (cm³) | | | Volume of the mask |
| | | Mask Volume of the | | | Volume of the mask associated to the |
| | | largest site (cm³) | | | largest site |
| | | Positive Mask | | | Volume of the positive voxels within |
| | | Volume (cm³) | | | the mask. |
| | | Positive Mask | | | Volume of the positive voxels within |
| | | Volume of the | | | the mask associated to the largest site |
| | | largest site (cm³) | | | |
| | | Surface Area (cm²) | | | External surface of the mesh. |
| | | Surface area of the | | | External surface of the largest site |
| | | largest site (cm²) | | | mesh |
| | | Mean surface to | | | Averaged of the ratios between area |
| | | volume ratio | | | and the volume of each site |
| | | Surface to volume | | | Ratio between area and the volume of |
| | | ratio of the largest | | | the largest site |
| | | site | | | |
| | | Mean compactness | | | Measures how compact or sphere-like |
| | | 1 | | | the volume is (averaged by site) |
| | | Compactness 1 of | | | Measures how compact or sphere-like |
| | | the largest site | | | the volume is. |
| | | Mean compactness | | | Measures how compact or sphere-like |
| | | 2 | | | the volume is (averaged by site) |
| | | Compactness 2 of | | | Measures how compact or sphere-like |
| | | the largest site | | | the largest site is. |
| | | Mean spherical | | | Measures how compact or sphere-like |
| | | disproportion | | | the volume is (averaged by site) |
| | | Spherical | | | Measures how compact or sphere-like |
| | | disproportion of the | | | the largest site is. |
| | | largest site | | | |
| | | Mean sphericity | | | Measures how compact or sphere-like |
| | | | | | the volume is (averaged by site) |
| | | Sphericity of the | | | Measures how compact or sphere-like |
| | | largest site | | | the largest site is. |
| | | Mean asphericity | | | Measures how compact or sphere-like |
| | | | | | the volume is (averaged by site) |
| | | Asphericity of the | | | Measures how compact or sphere-like |
| | | largest site | | | the largest site is. |
| | | Mass Center shift | | | The distance between the ROI volume |
| | | (cm) | | | centroid and the intensity-weighted |
| | | | | | ROI volume |
| | | Mean mass center | | | Averaged distance between each site |
| | | shift (cm) | | | volume centroid and the intensity- |
| | | | | | weighted site volume |
| | | Mass center shift of | | | The distance between the largest site |
| | | the largest site (cm) | | | volume centroid and the intensity- |
| | | | | | weighted largest site volume |
| | | Mean slice | | | The maximum 2D diameter is the |
| | | maximum 2D | | | maximum distance between the two |
| | | diameter (cm) | | | most distant points of the mesh |
| | | | | | projected on each slice of the image |
| | | | | | grid. (averaged by site) |
| | | Slice maximum 2D | | | The maximum 2D diameter is the |
| | | diameter of the | | | maximum distance between the two |
| | | largest site (cm) | | | most distant points of the mesh |
| | | | | | projected on each slice of the image |
| | | | | | grid. (largest site only) |
| | | Mean maximum 3D | | | The maximum 3D diameter is the |
| | | diameter (cm) | | | distance between the two most distant |
| | | | | | vertices in the ROI mesh vertex set. |
| | | | | | (averaged by site) |
| | | Maximum 3D | | | The maximum 3D diameter is the |
| | | diameter of the | | | distance between the two most distant |
| | | largest site (cm) | | | vertices in the ROI mesh vertex set |
| | | | | | (largest site only) |
| | | Mean major axis | | | The major axis length is twice the |
| | | length (cm) | | | largest semi-axis length of the |
| | | | | | ellipsoid, determined using the largest |
| | | | | | eigenvalue obtained by PCA on the |
| | | | | | mask voxels. (averaged by site) |
| | | Major axis length of | | | The major axis length is twice the |
| | | the largest site (cm) | | | largest semi-axis length of the |
| | | | | | ellipsoid, determined using the largest |
| | | | | | eigenvalue obtained by PCA on the |
| | | | | | mask voxels. (largest site only) |
| | | Mean minor axis | | | The minor axis length is twice the |
| | | length (cm) | | | second largest semi-axis length of the |
| | | | | | ellipsoid, determined using the second |
| | | | | | largest eigenvalue obtained by PCA |
| | | | | | on the mask voxels. (averaged by site) |
| | | Minor axis length of | | | The minor axis length is twice the |
| | | the largest site (cm) | | | second largest semi-axis length of the |
| | | | | | ellipsoid, determined using the second |
| | | | | | largest eigenvalue obtained by PCA |
| | | | | | on the mask voxels. (largest site only) |
| | | Mean least axis | | | The least axis length is twice the |
| | | length (cm) | | | smallest semi-axis length of the |
| | | | | | ellipsoid, determined using the |
| | | | | | smallest eigenvalue obtained by PCA |
| | | | | | on the mask voxels. (averaged by site) |
| | | Least axis length of | | | The least axis length is twice the |
| | | the largest site (cm) | | | smallest semi-axis length of the |
| | | | | | ellipsoid, determined using the |
| | | | | | smallest eigenvalue obtained by PCA |
| | | | | | on the mask voxels. (largest site only) |
| | | Mean inverse | | | Measures the extent to which a |
| | | Elongation | | | volume is longer than it is wide. |
| | | | | | (averaged by site) |
| | | Inverse Elongation | | | Measures the extent to which a |
| | | of the largest site | | | volume is longer than it is wide. |
| | | | | | (largest site only) |
| | | Mean inverse | | | Measures the extent to which a |
| | | Flatness | | | volume is flat relative to its length. |
| | | | | | (averaged by site) |
| | | Inverse flatness of | | | Measures the extent to which a |
| | | the largest site | | | volume is flat relative to its length. |
| | | | | | (largest site only) |
| | | Volume density - | | | Measures the proportion of the mesh |
| | | aligned bounding | | | axis-aligned bounding box volume |
| | | box | | | that is filled by the ROI volume. |
| | | Mean volume | | | Measures the proportion of the mesh |
| | | density - aligned | | | axis-aligned bounding box volume |
| | | bounding box | | | that is filled by the ROI volume. |
| | | | | | (averaged by site) |
| | | Volume density - | | | Measures the proportion of the mesh |
| | | aligned bounding | | | axis-aligned bounding box volume |
| | | box of the largest | | | that is filled by the ROI volume. |
| | | site | | | (largest site only) |
| | | Mean area density - | | | Measures the ratio between the ROI |
| | | aligned bounding | | | surface area and the axis-aligned |
| | | box | | | bounding box surface area. (averaged |
| | | | | | by site) |
| | | Area density - | | | Measures the ratio between the ROI |
| | | aligned bounding | | | surface area and the axis-aligned |
| | | box of the largest | | | bounding box surface area. (largest |
| | | site | | | site only) |
| | | Volume density - | | | Measures the proportion of the mesh |
| | | oriented bounding | | | oriented bounding box volume that is |
| | | box | | | filled by the ROI volume. |
| | | Mean volume | | | Measures the proportion of the mesh |
| | | density - oriented | | | oriented bounding box volume that is |
| | | bounding box | | | filled by the ROI volume. (averaged |
| | | | | | by site) |
| | | Volume density - | | | Measures the proportion of the mesh |
| | | oriented bounding | | | oriented bounding box volume that is |
| | | box of the largest | | | filled by the ROI volume. (largest site |
| | | site | | | only) |
| | | Mean area density - | | | Measures the ratio between the ROI |
| | | oriented bounding | | | surface area and the oriented bounding |
| | | box | | | box surface area. (averaged by site) |
| | | Area density - | | | Measures the ratio between the ROI |
| | | oriented bounding | | | surface area and the oriented bounding |
| | | box of the largest | | | box surface area. (largest site only) |
| | | site | | | |
| | | Volume density - | | | Measures the proportion of the mesh |
| | | enclosing ellipsoid | | | approximate enclosing ellipsoid |
| | | | | | volume that is filled by the ROI |
| | | | | | volume. |
| | | Mean volume | | | Measures the proportion of the mesh |
| | | density - enclosing | | | approximate enclosing ellipsoid |
| | | ellipsoid | | | volume that is filled by the ROI |
| | | | | | volume. (averaged by site) |
| | | Volume density - | | | Measures the proportion of the mesh |
| | | enclosing ellipsoid | | | approximate enclosing ellipsoid |
| | | of the largest site | | | volume that is filled by the ROI |
| | | | | | volume. (largest site only) |
| | | Mean area density - | | | Measures the ratio between the ROI |
| | | enclosing ellipsoid | | | surface area and the approximate |
| | | | | | enclosing ellipsoid surface area. |
| | | | | | (averaged by site) |
| | | Area density - | | | Measures the ratio between the ROI |
| | | enclosing ellipsoid | | | surface area and the approximate |
| | | of the largest site | | | enclosing ellipsoid surface area. |
| | | | | | (largest site only) |
| | | Volume density - | | | Measures the proportion of the mesh |
| | | convex hull | | | convex hull volume that is filled by |
| | | | | | the ROI volume. |
| | | Mean volume | | | Measures the proportion of the mesh |
| | | density - convex | | | convex hull volume that is filled by |
| | | hull | | | the ROI volume. (averaged by site) |
| | | Volume density - | | | Measures the proportion of the mesh |
| | | convex hull of the | | | convex hull volume that is filled by |
| | | largest site | | | the ROI volume. (largest site only) |
| | | Mean area density - | | | Measures the ratio between the ROI |
| | | convex hull | | | surface area and the convex hull |
| | | | | | surface area. (averaged by site) |
| | | Area density - | | | Measures the ratio between the ROI |
| | | convex hull of the | | | surface area and the convex hull |
| | | largest site | | | surface area. (largest site only) |
| | | Integrated intensity | | | The average intensity in the ROI, |
| | | | | | multiplied by the volume. |
| | | Mean integrated | | | The average intensity in the ROI, |
| | | Intensity | | | multiplied by the volume. (averaged |
| | | | | | by site) |
| | | Integrated Intensity | | | The average intensity in the ROI, |
| | | of the largest site | | | multiplied by the volume. (largest site |
| | | | | | only) |
| | | Maximum distance | | | Measures the distance between the |
| | | between mass | | | two most distant site mass centers. |
| | | centers (cm) | | | Measures the diffusion of a disease |
| | | | | | within an organ. |
| | | Mass center | | | The coordinates of the whole volume |
| | | coordinates (cm) | | | mass center. |
| | | Mean mass center | | | The averaged coordinates of each site |
| | | coordinates (cm) | | | mass center. |
| | | Mass center | | | The coordinates of the largest site |
| | | coordinates of the | | | mass center. |
| | | largest site (cm) | | | |

In a possible embodiment, extracting a bank of radiomics features from the connected components comprises calculating a summarized feature from the totality of the connected components to produce a summarized radiomics feature value; and storing the summarized feature value into the bank of radiomics features.

In a possible embodiment, extracting a bank of radiomics features from the connected components comprises calculating a component-specific radiomics feature value from each of the connected components; averaging the component-specific radiomics feature values to produce an average radiomics feature value; and integrating the average feature value into the bank of radiomics features.

In a possible embodiment, extracting a bank of radiomics features from the connected components comprises measuring a size of each connected component to identify the largest connected component; calculating a component-specific feature for the largest component to produce a maximal radiomics feature value; and integrating the maximal feature value into the bank of radiomics features.

In a possible embodiment, extracting a bank of radiomics features from the connected components comprises:
calculating a radiomics feature value from the totality of the connected components to produce a first value of this radiomics feature;
calculating a component-specific feature value from each of the connected components and averaging the component-specific feature values to produce a second value of the radiomics feature;
measuring a size of each connected component to identify the largest connected component, and selecting the component-specific feature of the largest component (calculated in the former step for averaging) to produce a third value of the radiomics feature;
storing the first, second and third values into the bank of radiomics features.

In a possible embodiment, extracting a bank of radiomics features from the connected components comprises:
For any of the non-morphological features, extracting a summarized feature from the totality of the connected components to produce a radiomics feature value;
For any of the morphological features:
   extracting a summarized feature from the totality of the connected components to produce a first value of the morphological feature;
   extracting a component-specific feature from each of the connected components;
   averaging the component-specific features to produce a second value of the morphological feature;
   measuring a size of each connected component to identify the largest connected component, and selecting the component-specific feature of the largest component to produce a third value of the morphological feature;
   integrating the first, second and third values of the morphological feature into the bank of radiomics features.

In one embodiment provided is a computer-implemented method for producing a bank of radiomics features from the radiological imaging data of a patient pathology having multiple lesion sites, the method comprising the steps of:
a) receiving radiological imaging data of the patient from a database of medical data;
b) localizing, from the radiological imaging data, at least one anatomical area of interest to produce an anatomical area label;
c) identifying, from the radiological imaging data, a set of imaging signal properties;
d) for each anatomical area label, a segmentation method for segmenting the radiological imaging data according to the imaging signal properties;
e) segmenting the radiological imaging data to produce at least two connected components, each connected component characterizing a lesion site, the connected components forming an object of interest to characterize a pathology of the patient;
f) extracting a bank of radiomics features from the at least two connected components;
g) storing the bank of radiomics features in a database of digital health features;
   characterized in that:
extracting a bank of radiomics features comprises a step of producing at least two values selected from: a total value, an average value and/or a maximum value of at least one radiomics feature calculated from all the connected components.

In further possible embodiment, it is proposed a computer-implemented method is described for producing a bank of radiomics features from the radiological imaging data of a patient, comprising:
a) receiving radiological imaging data of the patient from a first database of medical data;
b) localizing, from the radiological imaging data, at least one anatomical area of interest to produce an anatomical area label;
c) identifying, from the radiological imaging, a set of imaging signal properties;
d) for each anatomical area label, selecting a segmentation method for segmenting the radiological imaging data according to the imaging signal properties;
e) segmenting, with the selected segmentation method, the radiological imaging data to produce one or more connected components collectively forming an object of interest to characterize a pathology of the patient;
f) extracting a bank of radiomics features from the connected components;
g) storing the bank of radiomics features in a second database of digital health features.

The steps a), b), c), d), e) may each be manual, semi-automated, or automated steps, separately or in combination. In a preferred embodiment, steps a) to e) are fully automated into a radiomics feature extraction system which takes into input radiological images from a first database of medical data and which outputs a bank of radiomics features in a second database of digital health features.

In a possible embodiment, localizing, from the radiological imaging data, at least one anatomical area of interest comprises acquiring a localization model trained to extract an anatomical area label from radiological imaging data and applying the localization model to the radiological imaging data to produce an anatomical area label.

In a possible embodiment, identifying, from the radiological imaging data, a set of imaging signal properties comprises acquiring a signal identification model trained to extract a set of imaging signal properties from radiological imaging data and applying the signal identification model to the radiological imaging data to produce a set of imaging signal properties.

The segmentation method may be a manual method, a semi-automated method, or an automated method. In a possible embodiment, the segmentation method is an automated method, comprising acquiring a segmentation model trained to extract from radiological imaging data one or more connected component and applying the segmentation model to the radiological imaging data to produce one or more connected components.

In a possible embodiment, the segmentation method produces a plurality of connected components and extracting a bank of radiomics features from the connected components comprises extracting a summarized feature from the totality of the connected components to produce a radiomics feature value; and integrating the radiomics feature value into the bank of radiomics features.

In a possible embodiment, the segmentation method produces a plurality of connected components and extracting a bank of radiomics features from the connected components comprises extracting a component-specific feature from each of the connected components; averaging the component-specific features to produce a radiomics feature value;
and integrating the radiomics feature value into the bank of radiomics features.

In a possible embodiment, the segmentation method produces a plurality of connected components and extracting a bank of radiomics features from the connected components comprises measuring a size of each connected component to identify the largest connected component; extracting a component-specific feature for the largest component to produce a radiomics feature value; and integrating the radiomics feature value into the bank of radiomics features.

In a possible embodiment, the radiomics feature is a morphological feature, the segmentation method produces a plurality of connected components, and extracting a bank of radiomics features from the connected components comprises extracting a summarized feature from the totality of the connected components to produce a first radiomics feature value; extracting a component-specific feature from each of the connected components; averaging the component-specific features to produce a second radiomics feature value; measuring a size of each connected component to identify the largest connected component, and selecting the component-specific feature of the largest component to produce a third radiomics feature value; integrating the first, second and third radiomics values into the bank of radiomics features.

In a possible embodiment, the patient pathology is a cancer and each connected component corresponds to a different tumor or metastasis site. In a possible embodiment, the pathology is a non-small cell lung cancer (NSCLC) at stage IV, the anatomical area of interest is lung, and at least one of the connected components corresponds to a lung tumor or metastasis. In a possible embodiment, the pathology is a non-small cell lung cancer (NSCLC) at stage IV, the anatomical area of interest is liver, and at least one of the connected components corresponds to a liver metastasis.

In a possible embodiment, the patient pathology is an infectious disease, for instance SARS-COV2, the anatomical area of interest is lung and at least one of the connected components corresponds to a different ground glass opacity site.

### Exemplary Applications

In a particular embodiment, the patient pathology is a cancer and the radiological imaging data include data on multiple cancer lesion sites. In particular, the patient s lesion of a diseased tissue may have uneven morphology or spread to other sites, and it is referred herein as a multi-site lesion.

For example, the patient s cancer lesion may have uneven morphology or spread to form metastatic cancer sites and it is referred herein as a multi-site lesion. Examples of multi-lesion cancers are the stage IV of non-small cell lung cancer with liver metastases. Each connected component corresponds to a different tumor or metastasis site. The patient radiological imaging data may comprise two exams, one for the lung, one for the liver. From the lung images, the radiomics analyzer labels the localized anatomical area as lung, and extracts the radiomics features from at least one of the connected component corresponding to a lung tumor or metastasis. From the liver images, the radiomics analyzer labels the localized anatomical area as liver, and extracts the radiomics features from at least one of the connected component corresponding to a liver metastasis.

In another particular embodiment, the patient pathology is an infectious disease and the radiological imaging data include data on multiple infectious lesion sites. In particular, the patient s lesion of a diseased tissue may have uneven morphology or spread to other sites, and it is referred herein as a multi-site lesion. For example, the patient s SARS-COV2 lung lesions may have uneven morphology through multiple ground glass opacity site sites and it is referred herein as a multi-site lesion. The patient radiological imaging data may comprise a lung imaging exam. From the lung images, the radiomics analyzer labels the localized anatomical area as lung, and extracts the radiomics features from at least one of the connected component corresponding to a ground glass opacity site.

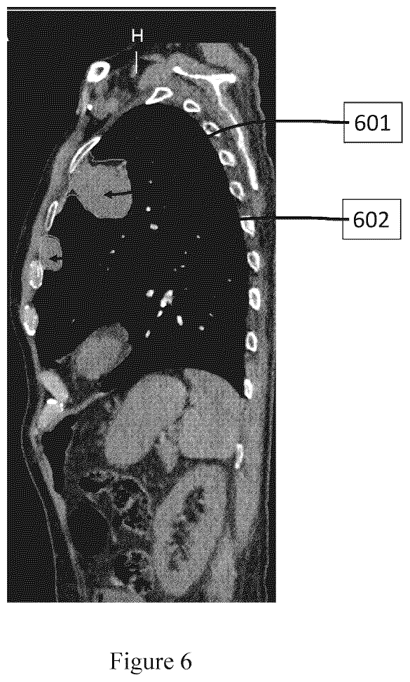

FIG. 6 illustrates an application of the proposed method to extract a bank of radiomics features from a lung cancer CT image. Automated segmentation produced 2 connected components 601 and 602 from a tumor ROI. The radiomics features are calculated according to the IBSI standard, for instance the mesh radiomics feature, the volume density radiomics feature (convex hull method), the integrated density as examples of morphological features in the IBSI standard. Instead of producing a single value for these IBSI features which strongly depend on the ROI morphology, the proposed method further extracts 2 values (average, max) or 3 values (Total, average, max) instead of only 1 summarized value (Global, default IBSI value). As can be seen in

Table 2, the resulting average and max figures are significantly different from the total value. By retaining two or three figures instead of just one, it is thus possible to produce an improved bank of radiomics features which summarizes with better accuracy the multisite properties of the patient radiomics image.

TABLE 2
Exemplary extended radiomics features extracted
from the radiological image of FIG. 6.

| IBSI feature | Total | Average | Max |
|---|---|---|---|
| Mesh (cm3) | 103 | 51.4 | 95.9 |
| Vol. density (convex hull) | 0.549 | 0.865 | 0.804 |
| Integrated intensity | 8.96 10\hat{ }4 | 4.36 10\hat{ }4 | 8.34 10\hat{ }4 |

This bank of radiomics features can then be employed for storage and later processing in longitudinal and/or multi-omics analysis workflows with more confidence in clinical practice, without requiring the need for manual verification, retrieval and manual processing of the radiomics image, segmentation, and IBSI feature extraction parametrization by a medical imaging expert. Note that this bank of feature also indifferently applies to a multi-site or to a single site ROI in the case where there is a single connected component, the total, the average and the mean values are simply the same radiomics feature value as can be calculated with a conventional radiomics feature extraction software of the prior art.

1. A computer-implemented method for producing a bank of radiomics features from the radiological imaging data of a patient pathology having multiple lesion sites, the method comprising the steps of:
a) receiving radiological imaging data of the patient from a database of medical data;
b) segmenting the radiological imaging data to produce at least two connected components, each connected component characterizing a lesion site, the connected components forming an object of interest to characterize a pathology of the patient;
c) extracting a bank of radiomics features from the at least two connected components;
d) storing the bank of radiomics features in a database of digital health features;
characterized in that:
extracting a bank of radiomics features comprises a step of producing at least two values selected from: a total value, an average value and/or a maximum value of at least one radiomics feature calculated from all the connected components.

2. The computer-implemented method of claim 1, further comprising the step of:
localizing, from the radiological imaging data, at least one anatomical area of interest to produce an anatomical area label.

3. The computer-implemented method of claim 2, further comprising the steps of:
acquiring a localization model trained to extract an anatomical area label from radiological imaging data; and
applying the localization model to the radiological imaging data to produce an anatomical area label.

4. The computer-implemented method of claim 3, further comprising the step of:
identifying, from the radiological imaging data, a set of imaging signal properties.

5. The computer-implemented method of claim 4, further comprising the steps of:
acquiring a signal identification model trained to extract a set of imaging signal properties from radiological imaging data; and
applying the signal identification model to the radiological imaging data to produce a set of imaging signal properties.

6. The computer-implemented method of claim 5, further comprising the step of:
selecting, for each anatomical area label, a segmentation method for segmenting the radiological imaging data according to the imaging signal properties.

7. The computer-implemented method of claim 6, wherein the segmentation method is a manual method, a semi-automated method, or an automated method.

8. The computer-implemented method of claim 7, wherein the segmentation method is an automated method, comprising the steps of:
acquiring a segmentation model trained to extract, from radiological imaging data, at least two connected components; and
applying the segmentation model to the radiological imaging data to produce at least two connected components.

9. The computer-implemented method of claim 7, wherein the segmentation method produces a set of at least two connected components and wherein extracting a bank of radiomics features from the connected components comprises:
aggregating the at least two connected components;
calculating a radiomics feature value from the aggregated connected components to produce a summarized radiomics feature value; and
storing the summarized radiomics feature value into the bank of radiomics features.

10. The computer-implemented method of claim 9, wherein the segmentation method produces a set of at least two connected components and wherein extracting a bank of radiomics features from the connected components comprises:
calculating a component-specific radiomics feature value from each component in the at least two connected components;
averaging the component-specific radiomics feature values to produce an average feature value; and
storing the average radiomics feature value into the bank of radiomics features.

11. The computer-implemented method of claim 10, wherein the segmentation method produces a set of at least two connected components and wherein extracting a bank of radiomics features from the connected components comprises:
measuring a size of each connected component in the at least two connected components to identify the largest connected component;
calculating a component-specific radiomics feature value from the largest connected component; and
storing the component-specific radiomics feature value into the bank of radiomics features.

12. The computer-implemented method of claim 7, wherein at least one of the radiomics features is a morphological feature, wherein the segmentation method produces at least two connected components and wherein extracting a bank of radiomics features from the connected components comprises:
aggregating all the connected components in the at least two connected components and calculating a summarized morphological feature from the aggregated connected components to produce a first morphological radiomics feature value;
calculating a component-specific morphological feature value from each component in the at least two connected components, and averaging the component-specific radiomics feature values to produce a second morphological radiomics feature value;
measuring a size of each connected component in the at least two connected components to identify the largest connected component, and calculating the component-specific morphological feature from the largest connected component to produce a third morphological radiomics feature value; and
storing the first, second and third morphological radiomics feature values into the bank of radiomics features.

13. The computer-implemented method of claim 12, wherein the patient pathology is a cancer, and each connected component corresponds to a different tumor or metastasis site.

14. The computer-implemented method of claim 13, wherein the pathology is a non-small cell lung cancer (NSCLC) at stage IV, the anatomical area of interest is lung, and at least one of the connected components corresponds to a lung tumor or metastasis.

15. The computer-implemented method of claim 13, wherein the pathology is a non-small cell lung cancer (NSCLC) at stage IV, the anatomical area of interest is liver, and at least one of the connected components corresponds to a liver metastasis.

16. The computer-implemented method of claim 12, wherein the patient pathology is an infectious disease, the anatomical area of interest is lung and at least one of the connected components corresponds to a different ground glass opacity site.

17. The computer-implemented method of claim 13, wherein the patient pathology is SARS-Cov2.

18. The computer-implemented method of claim 17, wherein the radiomics feature is a morphological radiomics feature selected among an IBSI feature or a pyradiomics feature.
